(19) United States
(12) Patent Application Publication
(10) Pub. No.: US 2022/0162545 A1
(43) Pub. Date: May 26, 2022

(54) COMPOSITIONS AND METHODS RELATED TO GROWING AND/OR PRODUCING BACTERIAL STRAINS USING AGRICULTURAL BYPRODUCTS

(71) Applicant: North Carolina Agricultural and Technical State University, Greensboro, NC (US)

(72) Inventors: Salam Ibrahim, Greensboro, NC (US); Tahl Zimmerman, Greensboro, NC (US)

(21) Appl. No.: 17/530,649

(22) Filed: Nov. 19, 2021

Related U.S. Application Data
(60) Provisional application No. 63/116,654, filed on Nov. 20, 2020.

(51) Int. Cl. C12N 1/20; C12N 1/04

(52) U.S. Cl. CPC C12N 1/20; C12N 1/04

(57) ABSTRACT

The presently disclosed subject matter relates generally to inexpensive and sustainable compositions and methods to support growth of certain bacteria. In particular, the presently disclosed methods and compositions do not rely on addition of enzymatic components to support the growth of bacteria.

### CROSS REFERENCE TO RELATED APPLICATIONS

This application claims priority pursuant to 35 U.S.C. § 119(e) to U.S. Provisional Patent Application Ser. No. 63/116,654 filed Nov. 20, 2020, which is hereby incorporated by reference in its entirety.

### STATEMENT AS TO RIGHTS TO INVENTIONS MADE UNDER FEDERALLY SPONSORED RESEARCH OR DEVELOPMENT

This invention was made with Government support under NI191445XXXXG007 awarded by the U.S. Department of Agriculture. The Government has certain rights in this invention.

### TECHNICAL FIELD

The presently disclosed subject matter relates generally to compositions including and/or for growing and/or producing bacterial strains such as, but not limited to lactic-acid producing bacteria (LAB), and to methods relating thereto such as methods to support the growth of bacteria including, but not limited to, LAB. In some embodiments, the presently disclosed methods and compositions do not rely on addition of enzymatic components to support the growth of bacteria.

### BACKGROUND

Research has explored effective production of various strains of bacteria, as the benefits to human health are better understood. For maximum benefit, growth of bacteria need yield a high cell count and high viability. Some research has focused on lactic-acid producing bacteria (“LAB”), with the express goal of increasing production of lactic acid; however, producing high amounts of lactic acid is at odds with the goal of producing high cell counts of LAB strains because the high quantities of lactic acid produced by LAB have also been found to inhibit growth of LAB strains.

Current growth methods for many strains of bacteria, including LAB strains, generally rely on the MRS media first reported by De Man J, Rogosa D and Sharpe M E ((1960) A medium for the cultivation of lactobacilli. Journal of Applied Bacteriology 23, 130-135). LAB require a variety of nitrogen sources; each of peptone, tryptone, beef extract and yeast extract are typically included in standard LAB growth media, including MRS. However, some of these nitrogen sources are expensive and some (e.g., beef extract) are animal-based products, which runs counter to certain cultural trends and/or dietary restrictions related to veganism and/or vegetarianism. In addition, the MRS media does not ultimately yield highly viable cells. Preparation of bulk-type media, such as MRS media, generally requires precise steps and trained individuals must be involved in the preparation of the starter cultures such a LAB, with careful attention to ensure the bacteria reach maximum growth levels, leading to high cell densities. The technology disclosed herein simplifies the preparation of media and achieves high cell densities.

Alternate growth media containing various agricultural products, for example corn steep liquor, tuna heads, pineapple peel juice, or wheat stillage+sugar beet molasses, have been employed, but the results have been limited. In particular, the use of agricultural products in growth media generally requires the addition of enzymes, which facilitate release of necessary nutrients from the referenced agricultural products; such nutrient-releasing enzymes include, but are not limited to, proteases, pectinases, cellulases, alpha-glucosidases, amylases and glucoamylases. Lactobacillus bulgaricus and Lactobacillus helveticus are each examples of LAB which produce nutrient-releasing enzymes (e.g., proteases and alpha-glucosidases), the production of which facilitate release of nutrients. Skim milk and whey-based media have been also used for bulk bacterial cell production; however, these media generally yield low cell density, as well as having cost and quality control issues.

Once bacteria are grown, poor long-term shelf stability of a variety of strains, including LAB, is an ongoing industry challenge and is generally addressed by “overdosing” (i.e. adding more starting material to a bacterial-containing product) so a product meets consumer expectations as well as label specifications of an identified live cell count after production and storage). Overdosing is wasteful and the health consequences of human consumption of products “overdosed” with bacteria remain unexplored.

Accordingly, there is still a need for compositions (e.g., growth media) and methods that can yield increased cell counts of bacteria, particularly for LAB and for improvements in shelf-stable bacterial compositions.

### SUMMARY

A first aspect of the present invention is directed to a method of preparing an extract, the method comprising: heating a composition comprising an agricultural by-product (e.g., waste sweet potatoes) and water to a temperature in a range of about 80° C. to about 110° C. for a period of time to provide a mixture comprising a liquid and solids; and isolating the liquid from the solids in the mixture, thereby providing the extract.

Another aspect of the present invention is directed to a method of preparing a bacterial growth media, the method comprising: combining an extract of the present invention (e.g., a sweet potato extract) and a base composition to thereby provide the bacterial growth media. In some embodiments, the method of preparing the bacterial growth media comprises preparing the extract according to a method of the present invention.

A further aspect of the present invention is directed to a composition comprising an extract (e.g., a sweet potato extract) and a base composition, wherein the base composition comprises a nitrogen source, a salt, an antioxidant and/or reducing agent, and an emulsifier. The extract may be prepared according to a method of the present invention.

Another aspect of the present invention is directed to a method of growing bacteria, the method comprising: culturing bacteria in a growth media to provide cultured bacteria, the growth media comprising a sweet potato extract and a base composition, wherein the base composition comprises a nitrogen source, a salt, an antioxidant and/or reducing agent, and an emulsifier. The extract may be prepared according to a method of the present invention.

These and other embodiments are described in greater detail in the detailed description which follows. An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a” and “an” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed subject matter can include the use of either of the other two terms.

As used herein, the term “about”, when referring to a value is meant to encompass variations of in one example ±20% or ±10%, in another example ±5%, in another example ±1%, and in still another example ±0.1% from the specified amount, as such variations are appropriate to practice the disclosed inventions. For example, “about X” where X is the measurable value, is meant to include X as well as variations of ±20%, ±10%, ±5%, ±1%, ±0.5%, or even ±0.1% of X.

In addition, all ranges disclosed herein are to be understood to encompass any and all subranges subsumed therein and any individual values therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. Further, recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

As used herein, phrases such as “between X and Y” and “between about X and Y” should be interpreted to include the values of X and Y. As used herein, phrases such as “between about X and Y” mean “between about X and about Y” and phrases such as “from about X to Y” mean “from about X to about Y.” Accordingly, all ranges disclosed herein are considered to include the end points of the range, unless expressly stated otherwise. For example, a range of “between 5 and 10”, “from 5 to 10” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

As used herein “allergen free” refers to a product (e.g., a composition) that contains less than 10% by mass of dairy, wheat, eggs, soy, shellfish, fish, tree nuts (including coconut) and/or peanuts, optionally as defined by the U.S. Food and Drug Administration. In some embodiments, allergen free refers to a product (e.g., a composition of the present invention) that contains less than 10% by mass of any referenced allergen (e.g., dairy, wheat, eggs, soy, shellfish, fish, tree nuts (including coconut) and/or peanuts) such as about 9%, 8%, 7%, 6%, 5%, 4%, 3%, 2%, 1% or 0% by mass of any referenced allergen. In some embodiments, allergen free refers to a product that contains less than 5% by mass, less than 2.5% by mass, less than 1% by mass, or less than 0.5% by mass of any referenced allergen. In some embodiments, allergen free refers to a product that contains about 0% by mass of any referenced allergen.

As used herein “animal product,” “animal-based” and “animal-derived product” as used herein interchangeably refer to a product (e.g., a compound or composition) that contains or is derived and/or obtained from an animal. Exemplary animal products include, but are not limited to, an animal-derived protein, beef extract, trypticase peptone, and/or casein or tryptone peptone such as from beef or milk. For example, MRS media is animal-based and contains about 10 g/L beef extract in 55 g/L media mix. Generally, “animal based” refers to a product that contains more than 20% by mass of an animal product such as about 19%, 18%, 17%, 16%, 15%, 14%, 13%, 12%, 11%, 10%, 9%, 8%, 7%, 6%, 5%, 4%, 3%, 2%, 1%, or more than 0% by mass of an animal product. In some embodiments, a composition of the present invention is devoid of an animal product and/or is not an animal-based composition.

As used herein “plant-based” refers to a product (e.g., a composition) that contains less than 20% by mass of an animal product. A plant-based composition may contain yeast or a yeast-based product, such as yeast extract and/or vegetable tryptone. In some embodiments, a plant-based product (e.g., a composition of the present invention) contains less than 20% by mass of an animal product such as about 19%, 18%, 17%, 16%, 15%, 14%, 13%, 12%, 11%, 10%, 9%, 8%, 7%, 6%, 5%, 4%, 3%, 2%, 1% or 0% by mass of an animal product. In some embodiments, plant-based refers to a product that contains less than 10% by mass, less than 5% by mass, less than 2% by mass, less than 1% by mass, or less than 0.5% by mass of an animal product. In some embodiments, a plant-based product refers to a product that contains about 0% by mass of an animal product.

As used herein “viability” is the ability of bacterial cells, such as LAB, to survive or live successfully. Bacterial viability is determined by measuring the number of live cells in a bacteria-containing composition. Generally, viability is determined by taking a bacteria-containing composition, such as a powder (e.g., including freeze-dried bacteria), putting the powder into solution (e.g., aqueous, buffered or peptone solution familiar to those of skill in the art, optionally to reconstitute the freeze-dried bacteria), diluting the solution according to standard methods, transferring the diluted solution to an agar plate, and allowing the bacterial population to grow for a length of time, generally about two days. The live bacteria on the agar plate can be manually counted using a Colony Counter (e.g., Bantex Colony Counter 920A) as Colony Forming Units (“CFU”).

As used herein, “stability” when referring to bacterial strains refers to the viability of bacterial cells in a composition (e.g., a product) over a period of time (e.g., 3, 6, 9, or 12 months or 1, 2, 3, or 4 years or more). In some embodiments, a composition has stability if live bacterial cells (optionally in an amount of at least about 10⁸ CFU/g for a powder or 10⁸ CFU/mL for a solution or broth or about 10⁹ CFU/g or 10⁹ CFU/mL) are found in the composition when measured at different points in time. Improved or higher stability generally refers to a higher viability measured for Sample A at one or more points in time compared to the viability of Sample B, measured at those same one or more points in time. In some embodiments, stability is measured for a composition of the present application near the end of cell growth, just before freeze drying, and/or after storage of the freeze dried samples, wherein such storage can be at room temperature (20-22° C.) or at 37° C. (the latter as an example of accelerated testing or stress testing for powdered, e.g., freeze-dried, bacterial compositions).

In some embodiments, bacteria cultured in a composition (e.g., media) of the present invention and/or according to a method of the present invention at a time near the end of cell growth and/or about 14, 15, or 16 hours of culturing in the composition have a cell count of at least about 6, 6.5, 7, 7.5, 8, 8.5, 9, or 9.5 log CFU/mL. In some embodiments, bacteria cultured in a composition (e.g., media) of the present invention and/or according to a method of the present invention have a cell count of at least about 6, 6.5, 7, 7.5, 8, 8.5, 9, or 9.5 log CFU/mL. In some embodiments, bacteria cultured in a composition (e.g., media) of the present invention and/or according to a method of the present invention at a time after preservation (e.g., freeze-drying) and/or storage for about 0.5, 1, 2, 3, 4, 5, 6, 7, 8, 9, 10, 11, or 12 months at room temperature (20-22° C.) or at 37° C. have a cell count of at least about 6, 6.5, 7, 7.5, 8, 8.5, 9, 9.5, 10, 10.5 or 11 log CFU/g. In some embodiments, a bacteria cultured in and freeze-dried with a composition of the present invention will have a cell count at least about 3 log CFU/g higher than the same strain of bacteria cultured and freeze-dried in an MSR media. In some embodiments, after 8 months of storage, a bacteria cultured in and freeze-dried with a composition of the present invention will have a cell count at least about 5.5 log CFU/g higher than the same strain of bacteria cultured and freeze-dried in an MSR media after 8 months of storage. A preserved bacteria (e.g., a freeze-dried bacteria) may be reconstituted prior to measuring amount of cells. In some embodiments, a preserved bacteria (e.g., a freeze-dried bacteria) is reconstituted by combining the preserved bacteria with water (optionally including a peptone and/or buffer), plating the composition including the reconstituted bacteria as described above and/or in accordance with known methods, and then counting the cells.

As used herein, the phrase “lactic-acid producing bacteria” or “LAB” refer to bacterial strains including, but not limited to, Lactobacillus casei, Lactobacillus gasseri, Lactobacillus rhamanos, Lactobacillus bulgaricus, Lactobacillus plantarum, Lactobacillus reuteri, and Lactobacillus acidophilus.

As used herein, “microflora” refers to an unwanted microorganism found in a living system such as the agricultural by-products described herein, including but not limited to waste sweet potatoes. Any microorganism other than bacteria targeted for growth using a composition (e.g., media) of the present application (e.g., a LAB such as L. casei, L. gasseri, L. rhamanos, L. bulgaricus, L. plantarum, L. reuteri, L. acidophilus and/or Bifidobacterium) are generally eliminated by heat treatment and/or a sterilization step that is designed to prevent the growth of those unwanted organisms, which can be found in the environment and/or are present in and/or on an agricultural by-product. Generally, waste sweet potatoes are rinsed before being cut, crushed, and/or peeled, wherein the rinsing removes soil, twigs, stones, and/or microflora that might be found on the surface of the potatoes.

“Waste potato” refers to a whole potato or potato parts that are deemed inedible or commercially undesirable. Whole potatoes can be deemed waste if they are, for example, split open, damaged, bruised, not within specification for size and/or shape. Waste whole potatoes are typically thrown away or even left unharvested in the ground. Potato parts can be deemed waste if they are excluded from the food production/consumption process, for example, potato peels or potato eyes that have been removed from whole potatoes by a food processor or restaurant. In one embodiment, waste potatoes are identified by one or more of a farmer, a food processor, a supermarket, or a restaurant. In some embodiments, potatoes can include sweet, red, white, yellow, fingerling, petite, blue/purple, and/or russet potatoes. In some embodiments, the waste potato comprises waste sweet potatoes. In some embodiments, waste sweet potato comprises sweet potato peels, which include sweet potato skin and a portion of the core of the sweet potato. In some embodiments, a sweet potato peel has a thickness of between about 0.1 inch and 2 inches; or between about 0.25 inch and 1.5 inches, or between about 0.5 inch and 1 inch; or about 0.75 inches. In some embodiments, a sweet potato peel comprises, by weight, less than 50% sweet potato core, less than 40% sweet potato core, less than 30% sweet potato core, less than 20% sweet potato core, or less than 10% sweet potato core. In some embodiments, waste sweet potato comprises sweet potatoes that have been cut or crushed. In some embodiments, waste sweet potato comprises sweet potato core in an amount of less than about 50%, 40%, 30%, 20%, 10%, 5%, or 1% by weight of the waste sweet potato.

As generally disclosed herein, a “base composition” comprises one or more (e.g., 1, 2, 3, or more) nitrogen source(s), one or more (e.g., 1, 2, 3, or more) salt(s), one or more (e.g., 1, 2, 3, or more) antioxidant(s) and/or reducing agent(s), and one or more (e.g., 1, 2, 3, or more) emulsifier(s), optionally in water. In some embodiments, a nitrogen source (e.g., yeast extract and/or a plant-based or bacterial-based peptone) may be present in a base composition in an amount of about 1, 1.5, 2, 2.5, or 3 g/L to about 3.5, 4, 4.5, 5, 5.5, 6, 6.5, 7, 7.5, 8, 8.5, 9, 9.5, or 10 g/L. In some embodiments, a base composition comprises a yeast extract in an amount of about 1 g/L to about 8 g/L, in an amount of about 2.5 g/L to about 7.5 g/L, or in an amount of about 4 g/L and/or a plant-based or bacterial-based peptone in an amount of about 1 g/L to about 8 g/L, in an amount of about 2.5 g/L to about 7.5 g/L, or in an amount of about 4 g/L. Exemplary salts that may be present in a base composition include, but are not limited to, magnesium-containing salts, manganese-containing salts (e.g., manganese sulfate), nitrogen-containing salts (e.g., ammonium phosphate), and/or buffering salts (e.g., potassium phosphate monobasic and/or potassium phosphate dibasic). In some embodiments, a base composition comprises one or more salt(s) in an amount of about 0.1, 0.2, 0.3, 0.4, 0.5, 0.6, 0.7, 0.8, 0.9, or 1 g/L to about 1.5, 2, 2.5, 3, 3.5, or 4 g/L, optionally in an amount of about 0.1 g/L to about 2.5 g/L, or in an amount of about 0.2 g/L to about 1 g/L. Exemplary antioxidants and/or reducing agents include, but are not limited to, cysteine and/or vitamin C (ascorbic acid). A base composition may comprise one or more antioxidant(s) and/or reducing agent(s) in an amount of about 0.1, 0.2, or 0.3 g/L to about 0.4, 0.5, 0.6, 0.7, 0.8, 0.9, 1, 1.5, 2, or 2.5 g/L, optionally in an amount of about 0.1 g/L to about 2.5 g/L, or in an amount of about 0.1 g/L to about 0.5 g/L. In some embodiments, a base composition comprises one or more emulsifier(s) in an amount of about 0.5, 1, 1.5 or 2 mL/L to about 2.5, 3, 3.5, 4, 4.5, 5, 5.5, 6, 6.5, 7, or 7.5 mL/L, optionally in an amount of about 1 mL/L to about 5 mL/L. Exemplary emulsifiers include, but are not limited to, emulsifiers that contain an oleic acid chain, such as Tween and/or SPAN, including, but not limited to, Tween 80, SPAN 20, SPAN 40, SPAN 80, and/or SPAN 85. A base composition of the present invention may be a solution.

In some embodiments, the presently disclosed subject matter provides a method of growing bacteria in a medium prepared from an agricultural by-product, the method comprising: (a) extracting nutrients from the agricultural by-product; (b) combining the extracted nutrients with one or more media component(s) (optionally a base composition) to provide a combined media; (c) culturing the bacteria in the combined media; and (d) isolating the cultured bacteria. In some embodiments, the one or more media component(s) do not include MRS media or added enzymes.

In some embodiments, the presently disclosed subject matter provides a method of preparing a media component using an agricultural by-product, comprising: (a) heating an agricultural by-product, such as waste sweet potatoes, in water to provide an aqueous mixture; (b) homogenizing the aqueous mixture to get a uniform suspension; (c) centrifuging the suspension; and (d) isolating the suspension fluid (extract) from the centrifuged sample; wherein the extract can be used as a media component for the growth of bacteria.

In some embodiments, the presently disclosed subject matter provides a method of preparing a media component using an agricultural by-product, comprising: (a) heating an agricultural by-product, such as sweet potato peels, in water to provide an aqueous mixture; (b) homogenizing the aqueous mixture to get a uniform suspension; (c) centrifuging the suspension; and (d) isolating the suspension fluid (extract) from the centrifuged sample; wherein the extract can be used as a media component for the growth of bacteria. A method of the present invention may extract one or more water soluble nutrient(s) from an agricultural by-product.

In some embodiments the agricultural by-product is a starch-rich agricultural byproduct, including but are not limited to potatoes, such as sweet, red, white, yellow, fingerling, petite, blue/purple, and russet potatoes, carrots, and/or unripened fruit. In some embodiments, the agricultural by-product is waste sweet potatoes such as sweet potato peels.

An extract of the present invention may be obtained from an agricultural by-product (e.g., waste potatoes such as waste sweet potatoes) as described herein. In some embodiments, the extract is a sweet potato extract. A sweet potato extract may be obtained according to a method of the present invention. In some embodiments, a sweet potato extract of the present invention is obtained from waste sweet potatoes, optionally from sweet potato peels. In some embodiments, an extract of the present invention comprises a water soluble nutrient such as, but not limited to, sugars (e.g., dextrose, glucose, fructose, sucrose and/or maltose) and sweet potato proteins (e.g., sporamines). Sweet potatoes (Table 1) generally have between about 1% and 3% protein content and sporamines account for more than 80% of the protein typically found in sweet potatoes. The specific concentration of sugars and proteins in the aqueous extract can vary, based on preparation and the amount of sweet potato or sweet potato peels used in the extraction. Sweet potatoes also contain insoluble fibers, such as cellulose, hemicellulose and lignin, and soluble fibers such as pectin.

TABLE 1
Nutrient content of uncooked raw sweet potatoes (77% water
content; 435 raw grams per grams per 100 g dry weight)

| Component | Amount |
|---|---|
| Nutrients | |
| Protein (g) | 7.0 |
| Fat (g) | 0.2 |
| Carbohydrates (g) | 87 |
| Fiber (g) | 13.0 |
| Sugar (g) | 18.2 |
| Minerals | |
| Calcium (mg) | 130 |
| Iron (mg) | 2.65 |
| Magnesium (mg) | 109 |
| Phosphorus (mg) | 204 |
| Potassium (mg) | 1465 |
| Sodium (mg) | 239 |
| Zinc (mg) | 1.30 |
| Copper (mg) | 0.65 |
| Manganese (mg) | 1.13 |
| Selenium (μg) | 2.6 |
| Vitamins | |
| Vitamin C (mg) | 10.4 |
| Thiamin (B1) (mg) | 0.35 |
| Riboflavin (B2) (mg) | 0.26 |
| Niacin (B3) (mg) | 2.43 |
| Pantothenic acid (B5) (mg) | 3.48 |
| Vitamin B6 (mg) | 0.91 |
| Folate Total (B9) (μg) | 48 |
| Vitamin A (IU) | 4178 |
| Vitamin E, alpha-tocopherol (mg) | 1.13 |
| Vitamin K1 (μg) | 7.8 |
| Beta-carotene (μg) | 36996 |
| Fats | |
| Saturated fatty acids (g) | 0.09 |
| Polyunsaturated fatty acids (g) | 0.04 |

In some embodiments, a bacterial strain present in a composition and/or for use in a method of the present invention is selected from the group of Bifidobacterium and LAB bacteria strains. In some embodiments, the Bifidobacterium and/or LAB do not produce nutrient-releasing enzymes such as, but not limited to, proteases, pectinases, cellulases, alpha-glucosidases, amylases and/or glucoamylases. In some embodiments, the Bifidobacterium and/or LAB do not produce a nutrient-releasing enzyme in a method and/or composition of the present invention. In some embodiments, the LAB do not produce pectinases, cellulases, amylases and/or glucoamylases. In some embodiments, the LAB bacteria is selected from the group of: L. casei, L. gasseri, L. rhamanos, L. bulgaricus, L. plantarum, L. reuteri, and L. acidophilus. In some embodiments, the bacteria is L. casei, L. gasseri, L. rhamanos, L. plantarum, L. reuteri, and L. acidophilus. In some embodiments, the bacteria is L. reuteri or L. bulgaricus. In some embodiments, the bacteria is L. reuteri. In some embodiments, the bacterial strains include L. reuteri (CF2F, DSM 20016, MM2-3, MM7) and/or L. bulgaricus (SR35).

In some embodiments, the bacteria grown according to a method of the present invention have a higher cell count than the cell count for the same bacteria grown in a MRS-based media under the same conditions. In some embodiments, bacteria grown according to a method of the present invention have an increased cell count compared to the cell count for the same bacteria grown in a MRS-based media under the same conditions, optionally wherein the increased cell count is between about 0.3 and about 1.1 log CFU/mL. In some embodiments, bacteria grown in a growth media of the present invention have a viability that is comparable to the viability of the same bacteria grown in a MRS-based media under the same conditions. In some embodiments, the viability is increased for bacteria grown in a growth media of the present invention compared to the viability of the same bacteria grown in a MRS-based media under the same conditions. Without being bound by theory, the increased buffering capacity of the growth media of the present application, 12.8+/−1.40 mL/1 pH value compared to 8.65+/−1.1 for the MRS broth may result in improved viability and long term stability. The improved buffering capacity of the growth media of the present application may also yield increased bacterial cell counts produced in the media as compared to the cell count of such bacteria produced in MRS media, such as lactic acid producing bacteria, as the growth is not inhibited by the lactic acid produced by the bacteria in the growth media of the present application.

In some embodiments, the presently disclosed subject matter provides a method of preparing a bacterial growth media comprising: (a) heating waste sweet potatoes, for example sweet potato peels, in an aqueous solution; (b) mixing the aqueous mixture to get a substantially uniform suspension; (c) isolating the suspension fluid from the suspension solids; and (d) combining the suspension fluid with a base composition.

In some embodiments, the presently disclosed subject matter provides a method of preparing a bacterial growth media, the method comprising: heating a composition comprising an agricultural by-product (e.g., waste sweet potatoes, such as sweet potato peels) and water to a temperature in a range of about 80° C. to about 100° C. for a period of time (e.g., about 1 minute to about 10 or 15 minutes) to provide a mixture comprising a liquid and solids; allowing the mixture to sit for some period of time (e.g. about 8 hours), separating the liquid from the solids present in the mixture to provide an extract; and combining the extract with an aqueous composition and/or base composition to thereby provide the bacterial growth media.

In some embodiments, the agricultural by-products are starch-rich agricultural byproducts. In some embodiments, the agricultural by-product is waste sweet potato. In some embodiments, the waste sweet potato is sweet potato peels, optionally containing less than 50% sweet potato core, less than 40% sweet potato core, less than 30% sweet potato core or less than 20% sweet potato core by weight of the waste sweet potato. In some embodiments, the agricultural by-products are waste sweet potatoes that have been cut or crushed. In some variations, the waste sweet potato is not heated before being combined with and/or added to water and/or an aqueous solution to provide a mixture; in some embodiments, the waste sweet potato is not baked before being combined with and/or added to water and/or an aqueous solution to provide a mixture.

In some embodiments, a growth media of the present invention does not include MRS media, an animal product (e.g., beef extract), and/or an added enzyme. In some embodiments, a growth media of the present invention does not include beef extract. In some embodiments it does not include an added enzyme. An “added enzyme” as used herein refers to an enzyme that is not naturally present in an extract of the present invention (e.g., a sweet potato extract). Extracts of the present invention are prepared from an agricultural by-product and thus may naturally include an enzyme from the agricultural by-product. In some embodiments of the present invention, no enzyme is added to an extract and/or a composition of the present invention; thus, the extract and/or composition may only include an enzyme that is naturally present in the extract. Accordingly, when a composition of the present invention is devoid of an added enzyme, this means that the composition does not include an enzyme that is not naturally present in the extract. In some embodiments, a growth media of the present invention does not include (is devoid of) MRS media. In some embodiments, a growth media of the present invention does not include (is devoid of) an animal product, such as beef extract. In some embodiments, a growth media of the present invention does not include (is devoid of) an added enzyme.

According to some embodiments, a method of preparing an extract is provided that comprises heating a composition comprising an agricultural by-product (e.g., waste sweet potatoes) and water to a temperature in a range of about 80, 85, or 90° C. to about 95, 100, 105, or 110° C. for a period of time (e.g., 1, 2, 3, 4, 5, 6, 7, 8, 9, 10, 11, 12, 13, 14, or 15 minutes) to provide a mixture comprising a liquid and solids; allowing the mixture to sit; and isolating the liquid from the solids in the mixture to provide the extract, wherein the liquid is the extract. In some embodiments, the agricultural by-product comprises and/or is sweet potato peels. The composition may comprise the agricultural by-product in an amount of about 10%, 15%, or 20% to about 25%, 30%, 35%, 40%, 45%, or 50% by weight of the composition. In some embodiments, heating the composition comprises heating the composition to a temperature in a range of about 80° C. to about 110° C. for about 1 minute to about 10 or 15 minutes. In some embodiments, the heating of the composition is under pressure, optionally a pressure of up to about 10, 15, or 20 psi. After heating, the mixture may be cooled, optionally to a temperature of about 4° C., and/or stored for a period of time (e.g., 1, 2, 3, 4, 5, 6, 7, 8, 9, 10, 11, 12, 13, 14, 15, 16, 17, 18, 19, 20, 21, 22, 23, or 24 hours or more). In some embodiments, after the heating step and prior to the isolating step, the mixture may be blended, optionally used an electric blender and/or a homogenizer. In some embodiments, the waste sweet potato and water mixture is blended, then heated, rapidly cooled, and stored overnight. Blending the mixture may provide a uniform mixture, optionally wherein the solids present in the blended mixture visually appear to be the same size and/or are substantially the same size (e.g., solids (e.g., particles) having a size within about ±20% of each other). In some embodiments, isolating the liquid from the solids in the mixture comprises obtaining the liquid and discarding the solids to thereby provide the extract. In some embodiments, isolating the liquid from the solids in the mixture comprises separating the liquid from the solids in the mixture. Methods of isolating and/or separating a liquid from a solid in a mixture are known in the art and include, but are not limited to, filtering, centrifuging, and/or allowing the solids to settle in the mixture and removing (e.g., decanting) the liquid from the mixture. In some embodiments, the extract (e.g., the liquid isolated from the mixture) is adjusted to a pH of about 5, 5.5, or 6 to about 6.5 or 7, optionally to a pH of about 6 to about 6.2. In some embodiments, the extract is heat treated (e.g., heated to about 80° C. for about 10 min) and/or sterilized, optionally to reduce and/or remove microflora that may be present in the extract. In some embodiments, the extract is a sweet potato extract. In some embodiments, the extract is prepared from waste sweet potatoes. In some embodiments, the extract is prepared from sweet potato peels.

In some embodiments, a method of preparing a composition of the present invention comprises combining an extract of the present invention and a base composition to thereby provide the composition of the present invention. The extract (e.g., a sweet potato extract) may be prepared according to a method of the present invention. In some embodiments, the method of preparing the composition comprises preparing an extract as described herein. The composition may be a media such as a growth media for bacteria. In some embodiments, the composition (e.g., growth media) comprises the extract and the base composition in a volume ratio of about 2:1 to about 1:20 (extract:base composition), optionally about 1:1, 1:2, 1:3, or 1:4. In some embodiments, the composition comprises the extract and the base composition in an amount of about 1:2 (extract:base composition). In some embodiments, a bacteria may be added to the composition (e.g., media) and the bacteria may be cultured and/or grown in the composition. After culturing and/or growing the bacteria in the composition, the bacteria may be dried and/or frozen (e.g., freeze-dried) and optionally stored.

A composition of the present invention that includes a base composition and an extract of the present invention may comprise a reduced amount of a nitrogen source (e.g., a yeast extract and/or a peptone) compared to the amount of a nitrogen source present in a MRS media. In some embodiments, the composition (including the base composition and extract) includes a nitrogen source in an amount that is about 20%, 30%, 40%, 50%, 60%, 70%, or 80% of the amount of a nitrogen source (e.g., the same nitrogen source) present in a MRS media. For example, a composition of the present invention (that includes a base composition and extract as described herein) may comprise a yeast extract and/or a peptone in an amount that is less than the amount of a yeast extract and/or a peptone present in a MRS media, optionally wherein the amount is about 50% or less than the amount present in the MRS media. In some embodiments, the composition (including the base composition and extract) is devoid of an animal product (e.g., beef extract), whereas the MRS media includes an animal product.

In some embodiments, preparation of a composition (e.g., an agricultural by-product media) of the present invention may include one or more of the following steps: sweet potato peels and/or waste sweet potatoes, as a representative agricultural by-product, are soaked in distilled water, e.g., at a weight/volume of 20-25%, the solution is heated (generally between about 90° C. and about 110° C. at up to about 15 psi), the pressure is reduced and the solution is allowed to sit and then cooled rapidly before being stored for at least about 8 hours, typically at 4° C. The components are mixed thoroughly and the aqueous solution, e.g., the extract, is isolated from the solids. The pH of the aqueous extract is adjusted to slightly acidic (pH<7.0), for example, around 5.9, 6.0, 6.1, 6.2, 6.3 or 6.4 The extract is then combined with a base composition, as disclosed herein, to yield a broth (e.g., media) for bacterial growth. Bacteria, such as lactic acid producing bacteria, may be cultured in the prepared broth, or media, under anaerobic conditions at 40° C. The bacteria produced in a broth of the present invention can then be prepared for storage and/or delivery, for example, using an emulsion system or drop method, spray drying or freeze drying.

In some embodiments, the rapidly cooled solution and/or mixture after heating can be stored at between about 2° C. and 10° C. for at least about 4 hours, 6 hours, 8 hours, 10 hours, 12 hours, 14 hours, 16 hours, 18 hours, 20 hours, 22 hours or 24 hours. In some embodiments, the solution is stored for up to 36 hours.

As disclosed herein, a sweet potato extract (SPE) may be combined with a base composition, yielding an SPE broth. In some embodiments, the ratio of SPE to base composition (SPE:base composition) ranges from about 2:1 to about 1:20, or from about 1:1 to about 1:10, or from about 1:1 to about 1:5, or from about 1:2 to about 1:3. In yet another embodiment, the ratio is about 1:2.

In some embodiments, bacterial cells are allowed to grow in the media of the present application until they reach at least about 1×10⁸ CFU/mL or 1×10⁹ CFU/mL. In some embodiments, a bacteria grown according to a method of the present invention may have a higher cell count than the cell count from growth of the same bacteria in an MRS-based media under the same conditions. In some embodiments, bacteria grown according to a method of the present application have about a 0.3 to about 1.1 log CFU/mL increase in the cell count compared to the same bacteria grown in an MRS-based media under the same conditions. In some embodiments, a bacteria grown in a media of the present invention may have a viability that is comparable to the viability of the same bacteria grown in MRS media under the same conditions; in some embodiments, the viability is greater than the viability of bacteria grown using MRS-based media.

In some embodiments, a composition of the present invention (e.g., a growth medium) does not contain (i.e., is devoid of) an animal product, such as beef extract. In some embodiments, the composition is allergen-free.

In some embodiments, a composition disclosed herein (e.g., an extract, base composition, and/or a growth medium) is sterilized according to a method familiar to one of skill in the art. For example, the composition can be heated at least about 100° C. or at least about 110° C. or at least about 115° C. or at least about 120° C. or at least about 125° C. for at least about 5 minutes, or at least about 10 minutes, or at least about 15 minutes, or at least about 20 minutes. Generally such sterilization occurs at a pressure of about 10, 15 or 20 psi.

In the embodiments, no enzyme is added to the bacterial growth medium of the present application to catalyze release of nutrients from the agricultural by-products. Such nutrient-releasing enzymes include, but are not limited to, proteases, pectinases, cellulases, alpha-glucosidasse, amylases, and/or glucoamylases. Some LAB are known to produce nutrient-releasing enzymes, e.g., proteases and alpha-glucosidases, which facilitate release of nutrients. In some embodiments, mechanical methods, not enzymatic methods, are used to release nutrients from starch-rich agricultural by-products, such as waste sweet potatoes.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

MRS broth was prepared following the instructions from the manufacturer: mixing 55 g of MRS media (Neogen) with 1 L of distilled water, followed by autoclaving at 121° C. for 45 minutes, then cooling to room temperature.

MRS contents (weight/volume)
Peptone 10.0 g/L
Yeast Extract 5.0 g/L
Beef Extract 10.0 g/L
Glucose 20.0 g/L
Potassium Phosphate 2.0 g/L
Sodium Acetate 5.0 g/L
Magnesium Sulfate 0.2 g/L
Manganese Sulfate 0.05 g/L
Tween 80 1.08 g/L
Ammonium Citrate 2.0 g/L

### Preparation of Sweet Potato Extract:

Sweet potatoes (Covington cultivar) were obtained from Burch Farms in Faison, N.C. For each experiment, 3 kg of fresh sweet potatoes were peeled at about ¾ inch, and the skins were collected and mixed with distilled water at 20-25% (w/v). The mixture was then boiled at 100° C. for 5 min up at about 15 psi, before the pressure was released, the solution rapidly cooled and was then stored overnight at 4° C.

In an alternate preparation, waste sweet potatoes (Covington cultivar) were obtained from Burch Farms in Faison, N.C. For each experiment, 3 kg of fresh sweet potatoes were cut up and mixed with distilled water at 20-25% (w/v). The mixture was then boiled at 100° C. for 5 min at about 15 psi, before being rapidly cooled and then stored overnight at 4° C.

The room temperature sweet potato peel aqueous mixture was homogenized to yield a uniform consistency, for example using homogenizer or a kitchen blender. The homogenized mixture was aliquoted into centrifuge tubes (50 ml) and the samples were centrifuged in a Sorvell RC 6 centrifuge (Fisher Scientific) for 30 min at 4° C. at 7,800 g. The suspension fluid was separated from the solids and adjusted to pH 6.00-6.20 using 0.1 N HCl. This fluid is referred to herein as “sweet potato extract” (SPE). Alternatively, the homogenized mixture can be filtered, the filtrate collected, and the pH adjusted as disclosed herein to produce the SPE. Those of skill in the art are familiar with methods for separating a solid from a liquid.

The SPE was then heated to 80° C. for 10 min in glass beakers held in a water bath; this heat treatment eliminates microflora from the broth.

A base solution was prepared from: yeast extract (4 g/L, Neogen), Bacto proteose peptone #3 (4 g/L, BD Biosciences), ammonium phosphate (1 g/L), manganese sulfate (0.2 g/L), potassium phosphate monobasic (1 g/L), potassium phosphate dibasic (1 g/L), cysteine (0.25 g/L) and 2-5 ml Tween 80; the components were combined in distilled water and sterilized at 121° C. for 15 minutes at 15 psi.

The SPE was then combined with the base solution at a ratio of 1:2 (SPE:base), yielding “SPE broth.”

### Bacterial Strains

Strains of Lactobacillus reuteri (CF2F, DSM 20016, MM7 and MM2-3, each obtained from Biogaia Biologics, Inc. Raleigh, N.C.) and Lactobacillus bulgaricus (SR35, from NIZO, The Netherlands) were used in this study. Each strain was cultured individually in 10 ml of MRS broth (Difco™ Lactobacilli MRS broth, Becton Dickinson and Co., Sparks, Md.) supplemented with 0.5 g/L of L-cysteine at 37° C. for 16 h.

### Fermentation Process:

Five milliliters of each of the bacterial strains were inoculated into 100 ml samples of the MRS broth or into 100 ml samples of the SPE broth. Each sample was then incubated at 38° C. up to 18 hr and periodically analyzed for pH values and bacterial population.

### Results

As shown in Table 2, the pH of L. reuteri in the SPE broth was similar to the pH of L. reuteri in MRS broth, the commercial standard, which indicates that the rate of growth was similar in the two media.

TABLE 2
pH values of L. reuteri incubated in MRS (control) or SPE broth

| | Fermentation Times | | |
|---|---|---|---|
| Strain/broth | 4 hours | 8 hours | 24 hours |
| CF2F | | | |
| MRS | 5.95 | 4.29 | 4.02 |
| SPE broth | 6.10 | 5.16 | 4.12 |
| DSM 20016 | | | |
| MRS | 6.10 | 5.23 | 4.11 |
| SPE broth | 6.19 | 5.46 | 4.07 |
| MM2-3 | | | |
| MRS | 5.29 | 4.98 | 3.90 |
| SPE broth | 6.04 | 4.55 | 4.10 |
| MM7 | | | |
| MRS | 6.13 | 4.46 | 4.27 |
| SPE broth | 6.06 | 4.51 | 4.17 |

As shown in Table 3, the L. bulgaricus and an L. reuteri strain showed the same yield from growth in the SPE broth compared to MRS broth (and at a fraction of the cost). Further, the stability of the bacterial strains grown in the SPE broth is higher after freeze drying compared to bacterial strains grown in MRS broth (Table 6).

TABLE 3
Cell counts (in log CPU/ml) of L. bulgaricus
strain SR35 and L. reuteri strain DSM 20016
incubated in MRS (control) or SPE broth

| Strain/broth | 0 hours | 8 hours | 16 hours |
|---|---|---|---|
| SR35 | | | |
| MRS | 3.00 | 4.25 | 9.21 |
| SPE broth | 3.00 | 4.19 | 8.90 |
| DSM 20016 | | | |
| MRS | 3.00 | | 7.60 |
| SPE broth | 3.00 | | 7.60 |

The MRS (control) and SPE broth described above comprise different nitrogen sources (see Table 4). Notably, the SPE broth contains less than half of each of proteose peptone #3 and yeast extract and no beef extract at all when compared to MRS.

TABLE 4
Nitrogen sources in each of MRS (control) and SPE broth

| Media | Nitrogen Sources | g/L Media |
|---|---|---|
| MRS broth | Proteose peptone #3 | 10 |
| | Beef Extract | 10 |
| | Yeast Extract | 5 |
| SPE broth | Proteose peptone #3 | 4 |
| | Ammonium phosphate | 1 |
| | Yeast extract | 4 |

### Example 2

Six strains of Lactobacillus were cultivated in MRS medium (prepared as described in Example 1) or in a plant-based medium of the present application at 42° C. for 14-16 hours until the stationary phase was reached, as determined by optical density using a spectrophotometer set to 600 nm. The number of colony forming units in each plate was determined by plating 100 μL samples on MRS/agar plates as disclosed herein. Generally, the samples were stored at −80° C. for 24 hours. Samples were optionally concentrated (e.g. reducing the volume from 100 mL to 60 mL) then freeze-dried in a lyophilizer and stored at room temperature over a period of 8 months.

### Yield

Yields are defined as the cell counts in cultures grown to the stationary phase. Six different strains of Lactobacillus were cultured in both the plant-based medium of the present application and MRS. The cell counts were determined after 14-16 hour growth, generally once the stationary phase was reached (Table 5). In all six cases, a higher cell count was achieved with the medium of the present application than the MRS medium, with the most dramatic difference being a nearly 1 log improvement in yield with Lactobacillus acidophilus and more than a 1 log improvement with Lactobacillus casei.

TABLE 5
Cell counts (in log CFU/ml) of bacterial strains after 14-16 hour
growth in 1 L plant-based medium or MRS medium (control)

| | | | Log increase in |
|---|---|---|---|
| | Plant-based | MRS | cell count |
| | Cell Count | Cell Count | for Plant-Based |
| Strain | (log CFU/ml) | (log CFU/ml) | over MRS |
| L. bulgaricus | 9.60 | 8.90 | 0.7 |
| L. casei subsp casei | 8.95 | 7.83 | 1.12 |
| L. gasseri | 9.15 | 8.67 | 0.48 |
| L. reuteri | 8.80 | 8.35 | 0.45 |
| L. plantarum | 8.47 | 8.16 | 0.31 |
| L. acidophilus | 8.90 | 7.95 | 0.95 |

### Stability

The effects on stability of culturing L. Bulgaricus in the plant based medium of the present application was compared to the MRS control at three time points: (i) immediately before freeze drying (ii) immediately after freeze drying and (iii) after a storage period of 8 months at room temperature (Table 6). Importantly, growing bacteria using the medium of the present application led to improved preservation of live cells after the freeze-drying step, compared to the destruction observed during freeze-drying of the cells cultured in MRS. After 8 months storage at room temperature of the freeze-dried material, the MRS grown cells had a >5 log reduction in number of log CFU/mL (9.5 to 4.3), while only ˜1 log cells were lost from the sample containing the medium of the present application. Without being bound by theory, the cells grown in the medium of the present application may be more robust and/or the components in the medium may function as a better preservative than MRS media during freeze-drying and storage.

TABLE 6
Survival of L. bulgaricus, as measured by log/CFU of bacteria

| | | | log increase in |
|---|---|---|---|
| | Plant-based | MRS | cell count |
| | Cell Count | Cell Count | for Plant-Based |
| Process Step | (log CFU/ml) | (log CFU/ml) | compared to MRS |
| immediately before | 10.89 | 9.5 | 1.39 |
| freeze drying | | | |
| immediately after | 10.41 | 7.1 | 3.31 |
| freeze drying | | | |
| after storage for | 9.8 | 4.3 | 5.5 |
| eight (8) months | | | |
| at room temperature | | | |

Thus, according to the methods disclosed herein, starch-rich agricultural by-products, including but not limited to sweet potato peels or waste sweet potatoes, can be used as part of successful growth media, yielding robust growth of lactic acid producing bacteria (LAB) comparable to the production of such bacteria using MRS media. Further, the media comprising sweet potato has been shown to increase the shelf stability of freeze dried LAB.

It will be understood that various details of the presently disclosed subject matter may be changed without departing from the scope of the presently disclosed subject matter. Furthermore, the foregoing description is for the purpose of illustration only, and not for the purpose of limitation.

That which is claimed is:

1. A method of preparing a bacterial growth media, the method comprising:
combining a sweet potato extract and a base composition, wherein the base composition comprises a nitrogen source, a salt, an antioxidant and/or reducing agent, and an emulsifier, to thereby provide the bacterial growth media.

2. The method of claim 1, further comprising preparing the sweet potato extract, wherein preparing the sweet potato extract comprises:
heating a composition comprising waste sweet potatoes and water to a temperature in a range of about 80° C. to about 110° C. for a period of time to provide a mixture comprising a liquid and solids; and
isolating the liquid from the solids in the mixture, thereby providing the sweet potato extract.

3. The method of claim 2, wherein the waste sweet potatoes comprise sweet potato peels.

4. The method of claim 2, wherein the composition comprises the waste sweet potatoes in an amount of about 10% to about 50% by weight of the composition.

5. The method of claim 2, wherein heating the composition comprises heating the composition to a temperature in a range of about 80° C. to about 100° C. for about 1 minute to about 10 or 15 minutes.

6. The method of claim 1, wherein the bacterial growth media comprises the sweet potato extract and the base composition in a volume ratio of about 1:1 to about 1:4 (sweet potato extract:base composition).

7. The method of claim 1, wherein the base composition comprises a yeast extract, a peptone, ammonium phosphate, manganese sulfate or potassium phosphate monobasic, potassium phosphate dibasic, cysteine and Tween 80.

8. A composition comprising:
a sweet potato extract; and
a base composition, wherein the base composition comprises a nitrogen source, a salt, an antioxidant and/or reducing agent, and an emulsifier.

9. The composition of claim 8, wherein the sweet potato extract is prepared by a method comprising:
heating a composition comprising waste sweet potatoes and water to a temperature in a range of about 80° C. to about 110° C. for a period of time to provide a mixture comprising a liquid and solids; and
isolating the liquid from the solids in the mixture, thereby providing the sweet potato extract.

10. A method of growing bacteria, the method comprising:
culturing bacteria in a growth media to provide cultured bacteria, the growth media comprising a sweet potato extract and a base composition,
wherein the base composition comprises a nitrogen source, a salt, an antioxidant and/or reducing agent, and an emulsifier.

11. The method of claim 10, wherein the sweet potato extract is prepared by a method comprising:
heating a composition comprising waste sweet potatoes and water to a temperature in a range of about 80° C. to about 110° C. for a period of time to provide a mixture comprising a liquid and solids; and
isolating the liquid from the solids in the mixture, thereby providing the sweet potato extract.

12. The method of claim 10, wherein the sweet potato extract and the base composition are present in the growth media in a volume ratio of about 1:1 to about 1:4 (sweet potato extract:base composition).

13. The method of claim 10, wherein the cultured bacteria produce at least about the same or more lactic acid compared to the amount of lactic acid produced by the same bacteria cultured in a media comprising an animal product (e.g., a MRS media) when cultured under the same conditions and measured at the same time during and/or following culturing of the bacteria.

14. The method of claim 10, wherein the growth media is devoid of an added enzyme and/or an animal product.

15. The method of claim 10, wherein the bacteria is selected from the group of Bifidobacterium and Lactobacillus strains.

16. The method of claim 10, wherein the bacteria is selected from the group of L. casei, L. gasseri, L. rhamanos, L. plantarum, L. reuteri, and L. acidophilus.

17. The method of claim 10, further comprising freeze-drying or spray drying the cultured bacteria to provide preserved bacteria.

18. The method of claim 16, wherein after culturing, the bacteria have a cell count of at least about 10⁸ CFU/mL.

19. The method of claim 17, wherein after freeze-drying, the bacteria have a cell count of at least about 10⁸ CFU/g.

20. A bacterial composition prepared according to the method of claim 11.
